SEQUENCE LISTING (11 sequences)

SEQ ID NO: 1 - DNA, 914 bp, Phoenix dactylifera
ggggggataccagttacccaaatcggccctctaagtatctgtgcgcaacggccagacatctttagaggccacttctgcgattcttgaagcgacccttgagagcattcatcgagtggtctccgtcctcacgcgtggatggctctcgagatcccccaatgcgacactctgagatcacctatcgactgggataccggtgataaaatgctcactggagccaagggatggaccttcttcccggaggttcagagatttcaggccatgcacataaataaatgcacgagttataaatatccacctcctctgaaggcgaaaatactgccgaagacttgcagtttgcttcccgctcatcccgcttcgttactctgcttcgaagtgcaaactggacaacaggtaagcgggggaaatggagggaacagggtatgcatagatttcggagattcgaacaaattggttggcactttagggttatacaatatatcttttccgttataagccttaatggaaggaaaggtgggggaaatccattttggattgagaaatcgatgtgccggtgcttaacttataatccagattgaaacttcgtgttcgtgatcaggacccagcaccaacaccagcgatgggggcgggcgacaataccaccatagcggttctagtagcagcaaaaaatagtggcataacaaacataagtagtagaacatgagtagcagaagtttacccggcgcaataaccggtggcactagtagcagaagcataggctttttgggtaccgggcagaggcaaggcatcgaatccctcatcaagcccatccacatcaatagctcactacccaccaacatcgaaataaggggatgagtcgactggcgaatcagctgcaataattggactcgaccagtgcaactggaacaacaggtaaa

SEQ ID NO: 2 - DNA, 363 bp, Phoenix dactylifera
ctctaagtatctgtgcgcaacggccagacatctttagaggccacttctgcgattcttgaagcgacccttgagagcattcatcgagtggtctccgtcctcacgcgtggatggctctcgagatcccccaatgcgacactctgagatcacctatcgactgggataccggtgataaaatgctcactggagccaagggatggaccttcttcccggaggttcagagatttcaggccatgcacataaataaatgcacgagttataaatatccacctcctctgaaggcgaaaatactgccgaagacttgcagtttgcttcccgctcatcccgcttcgttactctgcttcgaagtgcaaactggacaacagg

SEQ ID NO: 3 - protein, 118 aa, Phoenix dactylifera
MALEIPQCDTLRSPIDWDTGDKMLTGAKGWTFFPEVQRFQAMHINKCTSYKYPPPLKAKILPKTCSLLPAHPASLLCFEVQTGQQVSGGNGGNRVCIDFGDSNKLVGTLGLYNISFPL

SEQ ID NO: 4 - DNA, 21 bp, Artificial Sequence
  SRY 150 F (primer sequence)
agcagtcagggaggcagatca

SEQ ID NO: 5 - DNA, 25 bp, Artificial Sequence
  SRY-245R (primer sequence)
ccccctagtaccctgacaatgtatt

SEQ ID NO: 6 - DNA, 21 bp, Artificial Sequence
  SRY-2F (primer sequence)
gaatattcccgctctccggag

SEQ ID NO: 7 - DNA, 20 bp, Artificial Sequence
  SRY-2R (primer sequence)
acctgttgtccagttgcact

SEQ ID NO: 8 - DNA, 32 bp, Artificial Sequence
  ADOP-32 (adapter sequence)
aatacgactcactatagggcggccgcccgggc

SEQ ID NO: 9 - DNA, 25 bp, Artificial Sequence
  ADOP-27 (adapter sequence)
cactatacccgccggcgggcccgct

SEQ ID NO: 10 - DNA, 20 bp, Artificial Sequence
  primer T7
aatacgactcactatagggc

SEQ ID NO: 11 - DNA, 26 bp, Artificial Sequence
  SRY-1R (primer sequence)
gggctgtaagttatcgtaaaaggagc

(19) United States
(12) Patent Application Publication
(10) Pub. No.: US 2016/0138119 A1
(43) Pub. Date: May 19, 2016

(54) SEX-DETERMINATION METHOD FOR DATE PALM

(71) Applicant: King Faisal University, Al-Ahsa (SA)

(72) Inventors: MOHEI EL-DIN SOLLIMAN, AL-AHSA (SA); SULIMAN ALI AL-KHATEEB, AL-AHSA (SA); ABDULLATIF A. AL-KHATEEB, AL-AHSA (SA); HEBA ALLAH ADEL, CAIRO (EG)

(21) Appl. No.: 14/541,106

(22) Filed: Nov. 13, 2014

(51) Int. Cl. C12Q 1/68; G01N 33/68

(52) U.S. Cl. CPC C12Q 1/6895; G01N 33/68; C12Q 2600/13; G01N 2333/415

(57) ABSTRACT

A sex-determination method for a date palm plant includes obtaining a sample of a date palm plant and determining a presence or absence of the date palm SRY gene (SEQ ID NO: 1) in the sample. The presence of the date palm SRY gene (SEQ ID NO: 1) in the sample is indicative that the sample is from a male date palm plant. Using the sex determination method for a date palm plant, the sex of date palm plants may even be determined when the date palm plants are still young, i.e., prior to flowering of the plants.

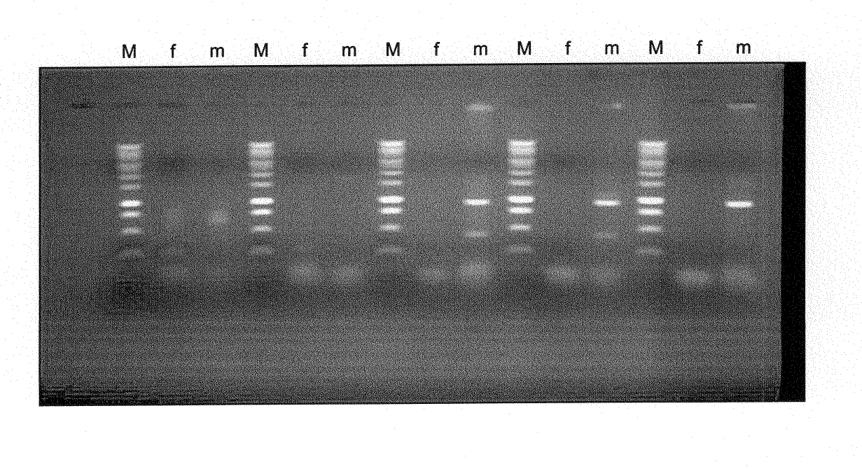

### INCORPORATION BY REFERENCE OF MATERIAL SUBMITTED IN COMPUTER READABLE FORM

The Applicants hereby incorporate by reference the sequence listing contained in the ASCII text file titled 32087_01_sequence_listing_ST25.txt, created Jun. 20, 2014 and having 4.72 KB of data (8.00 KB on disk).

### BACKGROUND OF THE INVENTION

1. Field of the Invention

The present invention relates to sex determination of the date palm (Phoenix dactylifera), and particularly to genetic sex determination of the date palm based on the first identification of the SRY gene in the date palm.

2. Description of the Related Art

The date palm (Phoenix dactylifera) is a dioecious plant, having separate male and female plants. Only the female plants of the date palm bear dates. While the male plants may be useful as pollinators, date palms are entirely pollinated manually in both traditional oasis horticulture and in the modern commercial orchards. As such, many date palm breeders and growers find little use in maintaining male plants. Thus, once the sex of the date palm plant is determined, many date palm breeders and growers proceed to maintain only the female date palm plants. The earliest point, however, at which male and female trees can be distinguished by external morphology is when the plant flowers, usually 5-8 years after planting. A method of determining the sex of the date palm plant at an earlier stage would avoid the need to invest time and expense in growing and maintaining unwanted male date palm plants.

Thus, a sex-determination method for date palm plants solving the aforementioned problem is desired.

### SUMMARY OF THE INVENTION

A sex-determination method for a date palm plant may include obtaining a sample of a date palm plant and determining a presence or absence of the date palm SRY gene (SEQ ID NO: 1) in the sample. The presence of the date palm SRY gene (SEQ ID NO: 1) in the sample is indicative that the sample is from a male date palm plant. Using the sex determination method for a date palm plant, the sex of date palm plants may be determined when the date palm plants are still young, i.e., prior to flowering of the plants.

Also provided are kits for sex determination of a date palm plant.

These and other features of the present invention will become readily apparent upon further review of the following specification and drawings.

### BRIEF DESCRIPTION OF THE DRAWING

The sole drawing FIGURE is a gel electrophoresis image of PCR amplification products obtained using SRY-150F primer (SEQ ID NO: 4) and SRY-245R primer (SEQ ID NO: 5).

Similar reference characters denote corresponding features consistently throughout the attached drawings.

### DETAILED DESCRIPTION OF THE PREFERRED EMBODIMENTS

A sex-determination method for a date palm plant may include obtaining a sample of a date palm plant and determining a presence or absence of the date palm SRY gene (SEQ ID NO: 1) in the sample. The presence of the date palm SRY gene (SEQ ID NO: 1) in the sample is indicative that the sample is from a male date palm plant. The sample may be obtained from any part of the date palm plant at any stage of development of the date palm plant. For example, the sample may include but is not limited to plant tissue (including leaves, seeds, petals, flowers, bark, etc.), extracts of plant tissue, and/or plant body fluid. Thus, using the sex determination method for a date palm plant, the sex of date palm plants may even be determined when the date palm plants are still young, i.e., prior to flowering of the plants.

The SRY (sex determining region Y) gene is a gene typically associated with initiation of male sex determination in humans, and is rarely discovered in plants. The present methods for sex-determination of a date palm plant are based on the surprising discovery by the present inventors of the date palm SRY gene (SEQ ID NO: 1). The present inventors have further identified SEQ ID NO: 2 as a fragment of the date palm SRY gene (SEQ ID NO: 1) which corresponds exactly to a fragment of the human SRY gene. Similar to the XY-system of chromosomes in humans, the male date palm SRY gene determines the gender of the offspring. Unlike humans, however, the sex chromosomes in the date palm are homomorphic.

Based on the discovery of the present inventors, the sex of a date palm plant may be genetically determined by determining a presence or absence of the date palm SRY gene (SEQ ID NO: 1) in a date palm plant sample. Determining a presence or absence of the date palm SRY gene (SEQ ID NO: 1) in the date palm plant sample may be carried out by any method known in the art. For example, determining a presence or absence of the date palm SRY gene (SEQ ID NO 1) in the date palm plant sample may include extracting nucleic acids from the sample, contacting under amplification conditions the nucleic acid from the sample with a male-specific date palm SRY primer pair, and detecting the presence or absence of amplification products or amplicons. The presence of amplification products may indicate the presence of the male-specific SRY gene in the date palm plant, i.e., that the sample is from a male plant. The absence of amplification products may indicate that the sample is from a female plant.

Alternatively, the presence or absence of the date palm SRY gene (SEQ ID NO: 1) in the sample may be determined by detecting a protein encoded by the date palm plant SRY gene (SEQ ID NO: 1) in the date palm plant sample using, for example, an enzyme-linked immunosorbent assay (ELISA). For example, detection of a protein including SEQ ID NO: 3 may indicate the presence of the date palm SRY gene (SEQ ID NO: 1) in the sample.

As discussed above, the nucleic acid extracted from the sample may be subjected to amplification conditions using male-specific SRY primer pairs. The male-specific SRY primer pairs may include primers specific for amplification of the date palm SRY gene (SEQ ID NO: 1). For example, the male-specific SRY primer pairs may include an oligonucleotide primer including SEQ ID NO: 4 and an oligonucleotide primer including SEQ ID NO: 5, or an oligonucleotide primer including SEQ ID NO: 6 and an oligonucleotide primer including SEQ ID NO: 7. The male-specific SRY primer pairs may include other primers specific for amplification of the date palm SRY gene (SEQ ID NO: 1). The presence of any amplification products may indicate the presence of a male-specific SRY gene in the date palm plant, i.e., that the date palm plant sample is from a male date palm plant. The presence of amplification products of a particular size may be further indicative of the presence of a male-specific SRY gene in the date palm plant. For example, amplification products having a size of approximately 800 base pairs may indicate that the date palm plant sample is from a male date palm plant.

One method for contacting under amplification conditions the nucleic acid from the sample with a male-specific date palm SRY primer pair may include subjecting the nucleic acid and the male-specific date palm SRY primer pair to standard polymerase chain reaction (PCR) cycles. The present inventors were able to determine the sex of different varieties of date palm varieties by conducting PCR screening for the presence of the SRY gene in different date palm plant varieties using a male-specific SRY primer pair. A gel electrophoresis image of PCR amplification products obtained using the oligonucleotide primer including SEQ ID NO: 4 and the oligonucleotide primer including SEQ ID NO: 5 is shown in the sole drawing figure, where (M) is a DNA1-KB Marker that was used, (m) is the male cultivar and (f) is the female cultivar.

The terms “nucleic acid” and “nucleic acid molecule” may be used interchangeably herein. The terms refer to a deoxyribonucleotide (DNA), ribonucleotide polymer (RNA), RNA/DNA hybrids and polyamide nucleic acids (PNAs) in either single- or double-stranded form, and unless otherwise limited, would encompass known analogs of natural nucleotides that can function in a similar manner as naturally occurring nucleotides.

Nucleic acid extracted from the sample may be isolated using known methods. Nucleic acid can be isolated using, for example, Plant DNAzol Reagent from Life Technologies now Invitrogen (Invitrogen Life Technologies), or DNeasy Mini-Kit (Qiagen). An isolated DNA sequence, for example, is substantially separated or purified away from other nucleic acid sequences with which the nucleic acid is normally associated in the cell of the organism in which the nucleic acid naturally occurs i.e., other chromosomal or extrachromosomal DNA. The term embraces nucleic acids that are biochemically purified so as to substantially remove contaminating nucleic acids and other cellular components. The term also embraces recombinant nucleic acids and chemically synthesized nucleic acids. The term “substantially purified”, as used herein, refers to a molecule separated from other molecules normally associated with it in its native state. More preferably, a substantially purified molecule is the predominant species present in a preparation. A substantially purified molecule may be greater than 60% free, preferably 75% free, more preferably 90% free from the other molecules (exclusive of solvent) present in the natural mixture. The term “substantially purified” is not intended to encompass molecules present in their native state.

It should be understood that, in addition to the techniques provided in the Examples herein, any suitable technique for extracting and/or isolating nucleic acids from biological samples that is known in the art may be used to extract nucleic acids and/or isolate nucleic acids from the date palm plant sample.

As is known in the art, PCR means a reaction for the in vitro amplification of a specific target nucleic acid sequence and is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. The reaction is cycled through different temperatures optimized for each step in a thermal cycler instrument. Particular temperatures, durations at each step, and rates of change between steps depend on many factors well own to those of ordinary skill in the art.

The term “PCR” further encompasses derivative forms of the reaction, including but not limited to, RT-PCR, real-time PCR, nested PCR, quantitative PCR, multiplexed PCR, and the like. “Reverse transcription PCR” or “RT-PCR” indicates a PCR that is preceded by a reverse transcription reaction that converts a target RNA to a complementary single stranded DNA, which is then amplified. For example, where RNA nucleic acid species may be used for detection of certain nucleotide sequences, a DNA copy (cDNA) of the RNA transcripts of interest can be synthesized prior to the amplification step. The cDNA copy can be synthesized by reverse transcription, which may be carried out as a separate step, or in a homogeneous reverse transcription-polymerase chain reaction, a modification of the polymerase chain reaction for amplifying RNA. “Real-time PCR” means a PCR for which the amount of reaction product, i.e. the amplicon or amplification product, is monitored as the reaction proceeds. There are many forms of real-time PCR that differ mainly in the detection chemistries used for monitoring the reaction product. “Nested PCR” means a two-stage PCR wherein the amplicon of a first PCR becomes the sample for a second PCR using a new set of primers, at least one of which binds to an interior location of the first amplicon. “Multiplexed PCR” means a PCR wherein multiple target sequences (or a single target sequence and one or more reference sequences) are simultaneously carried out in the same reaction mixture.

Any other suitable method for polynucleotide amplification that is well known to one of ordinary skill in the art may be employed. Other amplification methods may include for example, ligase chain reaction (“LCR”) and rolling circle amplification (“RCA”).

Any well-known methods for detection of amplification products may be employed. In some embodiments, the detection step can comprise gel electrophoresis, capillary electrophoresis, fluorescence resonant energy transfer (FRET), or hybridization to a labeled probe, such as a probe labeled with biotin, a fluorescent moiety, an antigen, a molecular weight tag, radioactive label, or other detectable modification. In some embodiments, the detection step can comprise the incorporation of a label (such as but not limited to fluorescent or radioactive labels) during an extension reaction. The detection step can further comprise measuring fluorescence, mass, charge, and/or chemiluminescence.

Real-time detection of the amplification products may be performed. Real-time detection in the context of amplification indicates an amplification reaction for which the amount of reaction product, i.e. the amplicon or amplification product, is monitored simultaneously with the reaction progression. Amplification products are monitored and quantitated as the amplification products are generated in the reaction mixture. Examples of real-time detection include RT-PCR (real-time polymerase chain reaction) and real-time quantitative PCR).

TaqMan PCR probes may be the basis for detection of the amplification products. TaqMan probes were developed by Applied Biosystems for use with real-time PCR reactions and are commercially available from Applied Biosystems. TaqMan probes comprise an oligonucleotide sequence containing a fluorophore covalently attached to the 5′-end of the oligonucleotide probe and a quencher at the 3′-end. Several different fluorophores (including. 6-carboxyfluorescein (FAM) or tetrachlorofluorescein (TET) and quenchers (e.g., tetramethylrhodamine (TAMRA) or dihydrocyclopyrroloindole tripeptide minor groove binder (MGB)) are available for inclusion in TaqMan probes. The quencher molecule quenches the fluorescence emitted by the fluorophore when excited by an appropriate light source via FRET (Fluorescence Resonance Energy Transfer). Upon extension of the TaqMan probes by Taq polymerase, the 5′ to 3′ exonuclease activity of the polymerase induces release of the fluorophore and breaks the close proximity to the quencher, thus relieving the quenching effect and allowing fluorescence of the fluorophore. Hence, fluorescence detected in the real-time PCR thermal cycler is directly proportional to the fluorophore released and the amount of DNA template present in the PCR.

In addition to the TaqMan fluorescent probes, other fluorescent probes may be used for detection of the amplification products. Fluorophores that may be used for fluorescent probes include but are not limited to DAPI (4′,6-dismidino-2-phylindole; FITC (fluorescein isothiocyanate), Dil (1,1′-dihexyl-3,3,3′,3′-tetramethlindocarbocyanine perchlorate), BODIPY FL and CY3, as well as any others commonly known to one of skill in the art.

As described above, determining a presence or absence of the date palm plant SRY gene (SEQ ID NO: 1) in the date palm plant sample may include detecting a protein encoded by the date palm plant SRY gene (SEQ ID NO: 1) using, e.g., an enzyme-linked immunosorbent assay (ELISA). For example, the protein encoded by the date palm plant SRY gene (SEQ ID NO: 1) may include SEQ ID NO: 3. The ELISA may be a direct ELISA in which antibodies to the protein of SEQ ID NO: 3 are produced, conjugated with enzymes, and applied directly to the sample prepared for testing. Detection of a binding complex between the protein (SEQ ID NO: 3) and the antibody indicates the presence of SEQ ID NO: 3. Preparation of antibodies and the extract from the date palm sample for use in the ELISA can be conducted in any suitable manner known in the art.

Also provided are kits for sex determination of a date palm plant. A kit for sex determination of a date palm plant may include one or more pairs of male-specific SRY primers and non-specific amplification reagents for amplifying the SRY gene. The male-specific SRY primer pairs may include SEQ ID NO: 4 and SEQ ID NO: 5 or SEQ ID NO: 6 and SEQ ID NO: 7. The kit may include a nucleic acid probe that binds to an amplified region of the SRY gene. The nucleic acid probe may be fluorescently labeled by any means known to one of ordinary skill in the art.

The kit for sex determination of a date palm plant may include an ELISA kit for use in detecting the presence of a protein encoded by SEQ ID NO: 1. The kit may include at least one antibody against the protein encoded by SEQ ID NO: 1 and at least one indicator to detect a binding complex of the protein encoded by SEQ ID NO: 1 and the at least one antibody.

The following examples are illustrative only, and are not intended to limit the present teachings.

### EXAMPLE 1

### Extraction of Plant DNA

Genomic DNA was extracted on a mini-prep scale as described by Murray and Thompson (1980) with some modifications. Small pieces of date palm leaf tissue (2.5 g) were frozen in liquid nitrogen and homogenized in 1000 μl of extraction buffer (2% CTAB, 1.4 M NaCl, 20 mM EDTA (pH 8.0), 100 mM Tris-HCl (pH 8.0), and 0.1 M β-Mercaptoethanol). The extract was incubated at 60° C. for 30 min. Phenol: chloroform: isoamyl alcohol (24:24:1; 500 μl) was added and mixed by vortexing for 30 seconds followed by centrifugation at 12,000×g for 5 minutes at room temperature. The aqueous phase was transferred to another tube and extracted again with 500 μl of chloroform: isoamyl alcohol (24:1) in an Eppendorf tube. Isopropanol was added at 0.6 volumes to the aqueous phase. Genomic DNA was precipitated, and the fibrous genomic DNA was spooled. Genomic DNA was then washed three times with 70% ethanol, dried in a vacuum, dissolved in TE containing 10 mg/ml RNase, and incubated at 37° C. for 30 mM. DNA was then extracted with phenol:chloroform:isoamyl alcohol, and the aqueous phase was transferred to a fresh tube. The genomic DNA was then precipitated by adding 0.3 M sodium acetate, pH 5.2 (final concentration) and 2.5 vol of ethanol. DNA was collected by centrifugation at 10,000×g for 20 min at 4° C. The pellet was washed with 70% ethanol, vacuum-dried, and dissolved in TE.

### EXAMPLE 2

### Spectrophotometric Estimation of Nucleic Acids

The quantity and quality of the nucleic acid were determined by measuring absorbance values at 260 nm and 280 nm. The absorbance for DNA was calculated with A260=50 μg/ml, and the absorbance for RNA was determined by A260=40 μg/ml. The purity of the nucleic acid was determined by calculating the ratio of A260/A280 for each sample.

### EXAMPLE 3

### Polymerase Chain Reaction

Taq polymerase, deoxyribonucleotide triphosphates (dNTPs), and convergent primers were used to amplify the DNA fragment. The reaction conditions for PCR involved denaturation at 94° C. for 30 seconds, annealing at 52° C. for 30 seconds, and extension at 72° C. for 60 seconds. After 35 cycles of amplification, an aliquot of the reaction mix was loaded onto a 0.8% agarose gel to examine the product.

### EXAMPLE 4

### Method for Early Identification of Gender in the Date Palm Plant

DNA samples from two female and three male plants were initially tested. Plants were from different cultivars in the Kingdom of Saudi Arabia (Shashi, Ruzeiz, Burhi, Khalas, and Sukari), more specifically from the Alhassa Oasis region. DNA was isolated according to Walsh et al. (1991).

Amplification of a male-specific SRY marker situated on the Y chromosome sex determination region was performed with a newly designed forward primer, SRY2 F 5-GAATATTCCCGCTCTCCGGAG-3 (SEQ ID NO: 6) and a reverse primer, SRY2R 5-ACCTGTTGTCCAGTTGCACT-3 (SEQ ID NO: 7). The newly designed SRY primers flanked an 800-bp region. Singleplex amplification was performed in a 25-Al volume containing 1 U DreamTaq Polymerase, 0.25 μM (0.5 μM, 0.75 μM, and 1.25 μM) of each primer, and 2.5 Al 10 dream buffer. The concentration of DNA varied from 0.25 ng to 10 ng. PCR reactions were performed for all of the templates. Samples were amplified through 35 cycles comprising 1 mm at 94° C., 30 s at 60° C., and 2 min at 72° C. following an initial denaturation at 95° C. for 10 min and final incubation at 72° C. for 10 min. In the next stage of analysis, a new primer pair binding to the SRY gene was co-amplified with the commercial PCR DreamTaq according to the technical manual. Separation and detection of alleles were performed by capillary electrophoresis.

### EXAMPLE 5

### DNA Sequencing

Sequencing of genes was performed by MACROGEN, Korea. Homology and structural comparison was made of the sequence of Date-SRY to the sequences of SRY genes in other species. Most sequence (DNA) analyses were performed with the CLCVector program and Genbank database. Homology searches were performed with FASTA. FASTA programs find regions of local or global similarity between Protein or DNA sequences, either by searching Protein or DNA databases, or by identifying local duplications within a sequence. Multiple sequence alignment was performed with CLUSTALW. CLUSTALW is a general purpose multiple sequence alignment program for DNA or proteins.

### EXAMPLE 6

### Isolation of SRY Gene

Restriction digestion: Date palm genomic DNA was restriction-digested overnight at 37° C. with 80 units of each enzyme in a final volume of 100 μL according to the manufacturer's instructions. Enzymes commonly used included BamHI and BglII Sou3A (Fermatas). Enzymes were heat-inactivated at 65° C. for 10 min and removed by extraction with an equal volume of chloroform-isoamyl alcohol (24:1 v/v). DNA was precipitated by adding 0.1 volumes of NaOAc (3 M [pH 5.2]) and two volumes of absolute ethanol. The mixture was vortexed, and the tubes were centrifuged at 13,200 rpm in a microcentrifuge for 30 min. The pellets were washed with 70% EtOH and centrifuged at 13,200 rpm for 10 min. Ethanol was aspirated from the pellet, and the pellet was air-dried. The pellet was then resuspended in 20 μL of sterile double-distilled water. The digested DNA was electrophoresed on a 0.8% agarose gel and further processed to digest the region from 0.5 to 1.0 kb. Each fragment was purified on Miniprep columns (Qiagen).

End-filling with Klenow: Digested DNA (50 μg) was partially end-filled by dGTP and dATP. The reaction mixture contained dNTPs (2 mM each), 10× reaction buffer (2.5 μl), and Klenow (2 μl) in a 50-μl volume and was incubated at 37° C. for 30 min. DNA was purified through Miniprep columns (Qiagen).

### EXAMPLE 7

### Adapter Annealing and Ligation

For adapter annealing and ligation, the following oligonucleotides were used:

ADOP-32
(SEQ ID NO: 8)
5′-AATACGACTCACTATAGGGCGGCCGCCCGGGC-3′;
and
ADOP-27
(SEQ ID NO: 9)
5′-CACTATACCCGCCGGCGGGCCCGCT-3.

Oligonucleotides ADOP-32 (SEQ ID NO: 8) and ADOP-27 (SEQ ID NO: 9) (synthesized by Macrogen, Korea) were resuspended in sterile double-distilled water at a concentration of 100 μM/μL. A volume of 20 μL of each adapter was pipetted into a 0.5-mL Eppendorf microfuge tube and overlaid with mineral oil. The adapters were heated at 99° C. for 4 mm in a beaker of water. Heat was removed, and the solution was allowed to cool for one hour at room temperature. The annealed adapters were decanted from the oil and stored at −20° C. Then, 10 μL of the date palm genomic restriction digest was ligated to 1 μL of the annealed adapters, 2 μL T4 DNA ligase buffer, and 2 μL T4 DNA ligase (5 units/μL; Life Promega) in a 20-μL reaction. The ligation was incubated overnight at 12° C. and heat-inactivated at 65° C. for 10 min. A 180-μL volume of TE (pH 8) was added to the ligation mix. This was called the adapter library.

Excess amounts of primer-adapter were removed by purification through QIAquik Columns (Qiagen).

Primary PCR amplification: The adapter-ligated genomic DNA was suitably diluted and used for the first PCR amplification in a 50-μl reaction volume in the following conditions: 2 min denaturation followed by a 35-cycle event of 30 s denaturation at 94° C., 30 s annealing at 48° C., 2 mm elongation at 72° C., and a final extension at 72° C. for 5 min.

Adapter primer T7 sequence 5′-AATACGACTCACTATAGGGC-3 (SEQ ID NO: 10) and SRY-3 (gene-specific primer, Date-SRY) were used for amplification. The sequence of the SRY-1R primer was 5′-GGGCTGTAAGTTATCGTAAAAGGAGC-3′ (SEQ ID NO: 11). Genomic DNA amplified with T7 (SEQ ID NO: 10) alone served as a negative control.

Secondary amplification: Amplified products (10 μl each) were electrophoresed on a 0.8% agarose gel and processed to assess amplification. The remaining sample of the primary amplification served as template after purification by phenol extraction. Precipitated DNA was resuspended in 20 μl TE. Various dilutions of the excised band were amplified with the T7 (SEQ ID NO: 10) and SRY-2R (SEQ ID NO: 7) primers. The sequence of the SRY-2 primer was SRY2R 5-ACCTGTTGTCCAGTTGCACT-3′ (SEQ ID NO: 7). The same reaction composition and cycle parameters were used.

### EXAMPLE 8

### Amplification of the Male-Specific SRY Marker

Amplification of the male-specific SRY marker situated on the Y chromosome sex determination (SRY) region was performed with a newly designed forward and reverse primers, SRY2 F 5-GAATATTCCCGCTCTCCGGAG-3 (SEQ ID NO: 6) and -SRY2R 5-ACCTGTTGTCCAGTTGCACT-3 (SEQ ID NO: 7). The newly designed SRY primers flanked an 800-bp region. Single amplification was performed in a 50 μl-Al volume containing 1 U DreamTaq Polymerase, 0.25 μM of each primer, and 5 μl 10× dream buffer. The concentration of DNA varied from 10 ng to 100 ng. PCR reactions were performed for all of the templates. Samples were amplified through 35 cycles comprising 30 s at 94° C., 30 s at 55° C., and 2 min at 72° C. following an initial denaturation at 95° C. for 5 min and final incubation at 72° C. for 10 min. Separation and detection of gene were performed by capillary electrophoesis.

It is to be understood that the present invention is not limited to the embodiments described above, but encompasses any and all embodiments within the scope of the following claims.

1. A sex-determination method for date palm, comprising:
obtaining a sample from a date palm plant;
extracting nucleic acid from the sample;
contacting under amplification conditions the nucleic acid from the sample with a male-specific date palm SRY primer pair for amplification of SEQ ID NO: 1; and
determining that the sex of the date palm plant is male if amplification products having an approximate size of at least 800 base pairs are detected.

2. The sex-determination method for date palm according to claim 1, wherein the sample includes date palm plant tissue or an extract from date palm plant tissue.

3. The sex-determination method for date palm according to claim 2, wherein the date palm plant tissue is selected from the group consisting of leaves, seeds, petals, flowers, and bark.

4. The sex-determination method for date palm according to claim 1, wherein the sample includes plant body fluid.

5. (canceled)

6. The sex-determination method for date palm according to claim 1, wherein the presence of amplification products having an approximate size of 800 base pairs indicates that the sex of the date palm plant is male.

7. The sex-determination method for date palm according to claim 1, wherein the male-specific SRY primer pair includes an oligonucleotide including SEQ ID NO: 4 and an oligonucleotide including SEQ ID NO: 5 or an oligonucleotide including SEQ ID NO: 6 and an oligonucleotide including SEQ ID NO: 7.

8. The sex-determination method for date palm according to claim 1, further comprising isolating the nucleic acid extracted from the sample.

9. The sex-determination method for date palm according to claim 1, wherein the nucleic acid is DNA.

10. The sex-determination method for date palm according to claim 1, wherein the nucleic acid is RNA.

11. The sex-determination method for date palm according to claim 1, wherein the amplification conditions comprise conditions for carrying out polymerase chain reaction (PCR).

12. A sex-determination method for date palm comprising:
obtaining a sample from a date palm plant;
detecting a protein in the sample, the protein being a protein that is encoded by SEQ ID NO: 1; and
determining that the sex of the date palm plant is male if the protein is detected.

13. The sex-determination method for date palm according to claim 12, wherein the protein is detected using an enzyme-linked immunosorbent assay (ELISA).

14. The sex-determination method for date palm according to claim 12, wherein the protein includes an amino acid sequence comprising SEQ ID NO: 3.

15. A kit for sex determination of a date palm plant, comprising:
a) a pair of male-specific SRY primers and amplification reagents for polymerase chain reaction (PCR) amplification of the SRY gene; or
b) at least one antibody against a protein encoded by SEQ ID NO: 1 and at least one indicator to detect a binding complex of the protein encoded by SEQ ID NO: 1 and the at least one antibody.

16. The kit for sex determination of a date palm plant according to claim 15, wherein the pair of male-specific SRY primers include an oligonucleotide including SEQ ID NO: 4 and an oligonucleotide including SEQ ID NO: 5 or an oligonucleotide including SEQ ID NO: 6 and an oligonucleotide including SEQ ID NO: 7.

17. (canceled)

18. The kit for sex determination of a date palm according to claim 15, wherein the protein encoded by SEQ ID NO: 1 includes the amino acid sequence of SEQ ID NO:3.
